(19) United States
(12) Patent Application Publication
(10) Pub. No.: US 2025/0188022 A1
(43) Pub. Date: Jun. 12, 2025

(54) PROCESS FOR THE PREPARATION OF NAFAMOSTAT, CAMOSTAT AND THEIR DERIVATIVES

(71) Applicant: COUNCIL OF SCIENTIFIC & INDUSTRIAL RESEARCH, New Delhi (IN)

(72) Inventors: Riyaz AHMED, Jammu (IN); Gulshan KUMAR, Jammu (IN); Sheena MAHAJAN, Jammu (IN); Praveen Kumar VERMA, Jammu (IN); Pankaj Singh CHAM, Jammu (IN); Amit KUMAR, Jammu (IN); Qazi Naveed AHMED, Jammu (IN); Dumbala Srinivasa REDDY, Jammu (IN); Ravi SHANKAR, Jammu (IN); Parvinder Pal SINGH, Jammu (IN)

(21) Appl. No.: 18/836,241

(22) PCT Filed: Feb. 7, 2023
(86) PCT No.: PCT/IN2023/050123

§ 371 Date: Aug. 6, 2024

(30) Foreign Application Priority Data
Feb. 7, 2022 (IN) 202211006599

(51) Int. Cl. C07C 277/08

(52) U.S. Cl. CPC C07C 277/08

(57) ABSTRACT

The invention provides a novel, economical and practical route for the synthesis of an ester from acid and alcoholic functionalities using Trihalotriazine as coupling agent. Specifically, the invention provides a novel, economical and practical route for the preparation of Nafamostat and Camostat. Synthesis of p-guanidinobenzoic acid (Al) was also achieved from thiourea and p-aminobenzoic acid by using trihalotriazine as coupling reagent.

### FIELD OF THE INVENTION

The present invention provides a process for compound of general Formula (F), or a salt or an isomer thereof. More particularly the present invention provides an effective and economical method for the preparation of highly functionalized ester based drugs namely Nafamostat, Camostat and their derivatives.

### BACKGROUND OF THE INVENTION

Nafamostat mesylate, also known as FUT-175 or 6′-amidino-2-naphthyl-4-guanidinobenzoate dihydrochloride, is a broad-spectrum serine protease inhibitor synthesized by Fujii et al. (Biochim Biophys Acta, 1981, 661, 342) and placed in market by Japan Tobacco in 1986 for the treatment of inflammatory-related diseases, such as pancreatitis (Surgery, 2001, 130, 175), disseminated intravascular coagulation (DIC), and systemic inflammatory response syndrome.

Another guanidine analogue, Camostat, originally known as FOY-305, was first reported by Ono Pharmaceutical Co., Ltd. In 1977 and its mesylate salt was approved in Japan in 1985 for the treatment of reflux esophagitis and chronic pancreatitis (Org Process Res Dev, 2020, 24, 940; Digestion, 2004, 30, 171;

Several methods have been published for the synthesis of Nafamostat and Camostat. Major strategies for the synthesis of Nafamostat and Camostat has been based on the preparation of fragments such as fragment A1 as p-guanidinobenzoic acid, fragment B1 as 6-hydroxy-2-naphthimidamide and fragment C1 as 2-(dimethylamino)-2-oxoethyl 2-(4-hydroxyphenyl) acetate and coupling of the two fragments (A1 and B1) for the preparation of Nafamostat and the coupling of two key fragments (A1 and C1) for the synthesis of Camostat.

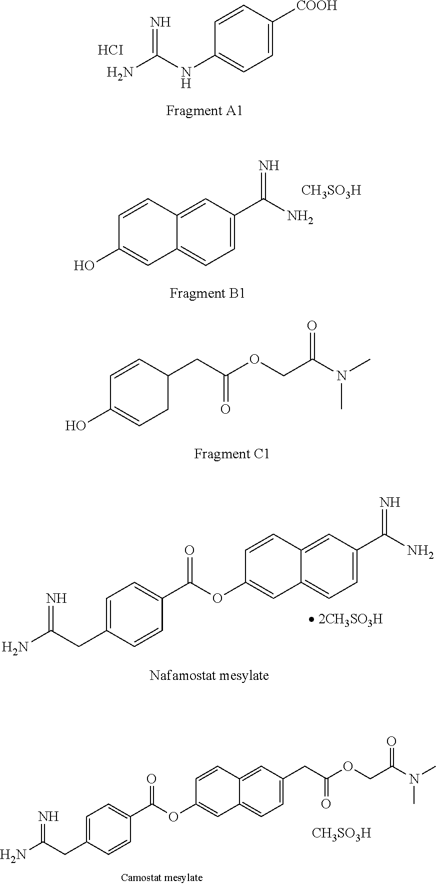

Most of the process described earlier followed the similar basic path for the preparation of fragments A1 and B1. The preparation of Nafamostat from fragments A1 and B1 require specific coupling agents.

CN103012214A described the preparation method of a Nafamostat mesylate hydrochloride from amidino-β-naphthol methane sulfonate and guanidine radicals benzoyl chloride hydrochloride salt at 0-5° C. in pyridine and then converted into Nafamostat mesylate in final step.

CN103641749B and CN103641749B in 2013 described the preparation method of Nafamostat mesylate from p-guanidinobenzoic acid hydrochloride, 6-amidino-β-naphthol using 4,5-dicyano imidazole (DCI) as coupling agent in methylene dichloride mix, 0-5° C. is stirred 1 hour, then improve temperature to 18-22° C., insulation reaction 8-12 h and then finally obtained as mesylate salt.

KR101595747B1 claimed the preparation method of Nafamostat mesylate from p-guanidinobenzoic acid hydrochloride and 6-amidino-2-naphthol methanesulfonate in the presence of N,N′-diisopropylcarbodiimide (DIC) and 4-dimethylaminopyridine (DMAP) in pyridine as coupling agent.

EP0465913B1 describes a method for preparing diaminotrifluoromethylpyrimidine derivatives using condensation agents such as dicyclohexylcarbodiimide (DCC), N,N′-carbonyldiimidazole (CDI), 1-ethyl-3-(3-dimethylaminopropyl) carbodiimide (EDC).

CN113999145A describes method for the preparation of Nafamostat from p-chlorobenzoic acid and 6-amidino-2-naphthol hydrochloride utilizing carbodiimide based DCC as coupling reagent.

EP0048433B1 also describes a method for preparing Nafamostat mesylate wherein DCC is reacted with 4-guanidinobenzoic acid hydrochloride, and then coupled with 6-amidino-2-naphthol methanesulfonate.

In the same way, another guanidine analogue Camostat also synthesized by using hazardous chemical or coupling reagents such as DIC, EDC, DCC etc.

Fuji et al. in 1977 has reported synthesis of Camostat mesylate in U.S. Pat. No. 4,021,472 by reacting the N,N-dimethylcarbamoylmethyl-p-hydroxybenzoate with the acid chloride derivative of p-guanidinobenzoic acid in pyridine at room temperature for 2 h. The crystals were produced by adding sodium bicarbonate solution, filtered and then dissolving in methanol and rendering it acidic with methanesulfonic acid. On adding diethyl ether Camostat mesylate precipitates out.

Most of the prior art methods regarding the synthesis of said molecules requires the coupling of fragment A1 and fragment B1 (or fragment C1) using carbodiimide based coupling reagent makes the process quite expensive and tedious work up.

Considering the importance of these drugs, effective coupling conditions with inexpensive and ease of operation is highly required. In this direction, the present invention provides an alternate and effective method for the synthesis of Nafamostat and Camostat utilizing the trihalotriazine as inexpensive and effective coupling reagent which also provides the additional advantage towards the ease of operation regarding work up.

### OBJECTIVE OF THE INVENTION

The objective of the invention is to provide a process for compound of general Formula (F).

Another objective of the invention is to provide a novel, practical and economical route for the preparation of Nafamostat and Camostat drugs and their derivatives.

Yet another objective of the invention is to explore trihalotriazine more preferably trichlorotriazine (TCT) as the novel and highly economical coupling agent for the preparation of esters and its application in synthesis of Nafamostat and Camostat drugs and their derivatives.

### SUMMARY OF THE INVENTION

Accordingly, the present invention provides a process for the preparation of compound of general Formula F

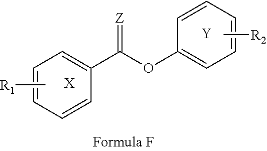

wherein Z is O, S, NH;

Ring X and Y is aryl, heteroaryl, extended rings selected from group consisting of naphthalene, phenanthrene, quinoline, isoquinoline;

R₁ is guanidinyl, amidinyl, 2-(dimethylamino)-2-oxoethyl acetyl, halogens, alkyl groups, amide group, cyano group, nitro group, amino group, methoxy group, O-benzyl esters, N-benzyl esters, hydroxyl group, aryl groups, heteroaryl groups;

R₂ is guanidinyl, amidinyl, 2-(dimethylamino)-2-oxoethyl acetyl, halogens, alkyl groups, amide group, cyano group, nitro group, amino group, methoxy group, O-benzyl esters, N-benzyl esters, hydroxyl group, aryl groups, heteroaryl groups, substituted benzenes;

wherein the said process comprises the steps;
i. reacting 2,4,6-trihalo-1,3,5-triazine and base at 25-40° C. for 5 minutes to obtain activated complex;
ii. reacting the activated complex as obtained in step-i with fragment A at 40-60° C. for 4 hours to obtain intermediate complex;
iii. reacting the intermediate compound as obtained in step-ii with fragment B in the ratio of ranging from 0-1 and with fragment C in the ratio of ranging from 0-1 to obtain compound D;
iv. treating the compound D as obtained in step-iii with aqueous sodium bicarbonate;
v. optionally purifying the obtained bicarbonate salt of compound D as obtained in step-iv;
vi. treating the compound D as obtained in step iv & v with methanesulfonic acid in presence of methanol to provide Formula F.

In an embodiment of present invention the representative compounds are selected from Nafamostat mesylate and Camostat mesylate.

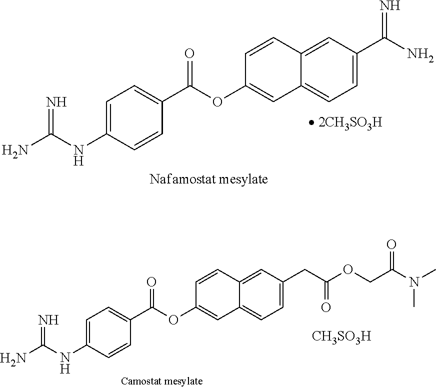

In another embodiment of present invention the fragment A is prepared by reacting p-aminobenzoic acid (1) with thiourea (4) in presence of base in ethanol.

In another embodiment of present invention the base is selected from the group consisting of pyridine (py), N-methylmorpholine (NMM) triethylamine (Et₃N) Diazabicycloundecene (DBU), more preferably N-methylmorpholine (NMM).

In another embodiment of present invention the trihalotriazine is selected from the group consisting of trichlorotriazine, tribromotriazine, trifluorotriazine.

In another embodiment of present invention the base is selected from sodium carbonate, cesium carbonate, ammonium carbonate and more preferably potassium carbonate.

In another embodiment of present invention the yield of Nafamostat mesylate is 54-70% and Camostat mesylate is 17-28%

In an embodiment of present invention the representative compounds are selected from

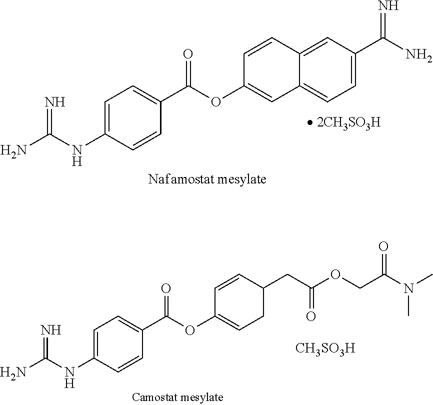

In another embodiment of present invention the base is selected from the group consisting of pyridine (py), N-methylmorpholine (NMM) triethylamine (Et₃N) Diazabicycloundecene (DBU), more preferably N-methylmorpholine (NMM).

In another embodiment of present invention the trihalotriazine is selected from the group consisting oftrichlorotriazine, tribromotriazine, trifluorotriazine.

### List of Abbreviations Used:

| | |
|---|---|
| 1) | TCT—Trichlorotriazine |
| 2) | NMM—4-methylmorphine |
| 3) | Et₃N—Triethylamine |
| 4) | DBU—Diazabicycloundecene |
| 5) | Py—Pyridine |
| 6) | DCC—N,N-Dicyclohexylcarbodiimide |
| 7) | DIC—N,N-Diisopropylcarbodiimide |
| 8) | EDC—1-Ethyl-3-(3-dimethylaminopropyl)carbodiimide |
| 9) | DCU—N,N-Dicyclohexylurea |
| 10) | DMAP—Dimethylaminopyridine |
| 11) | CuCN—Copper cyanide |
| 12) | DMF—Dimethyformamide |
| 13) | H₂O₂—Hydrogen peroxide |
| 14) | Br₂—Bromine |
| 15) | Sn—Tin |
| 16) | AcOH—Acetic acid |
| 17) | H₂O—Water |
| 18) | HBr—Hydrogen bromide |
| 19) | HCl—Hydrochloric acid |
| 20) | CH₃OH—Methanol |
| 21) | NH₃—Ammonia |
| 22) | MSA—Methanesulfonic acid |
| 23) | CH₃CO₃H—Peracetic acid |
| 24) | K₂CO₃—Potassium carbonate |
| 25) | DCM—Dichloromethane |
| 26) | ACN—Acetonitrile |
| 27) | EtOH—Ethanol |
| 28) | DMSO-d⁶—Deuterated dimethylsulfoxide |
| 29) | CDCl₃—Deuterated chloroform |
| 30) | CD₃OD—Deuterated methanol |
| 31) | NMR—Nuclear Magnetic Resonance |
| 32) | PPM—Parts Per Million |
| 33) | TLC—Thin Layer Chromatography |
| 34) | THF—Tetrahydrofuran |
| 35) | Spectrometry |
| 36) | MHz—Megahertz |

### BRIEF DESCRIPTION OF DRAWINGS

FIG. 1 depicts the scheme for the process for preparation of Nafamostat, Camostat and their derivatives

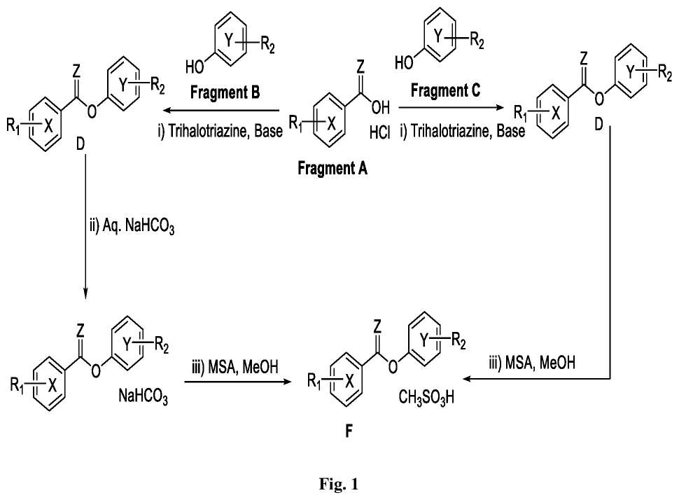

FIG. 2 depicts ¹H-NMR of Nafamaostat mesylate.

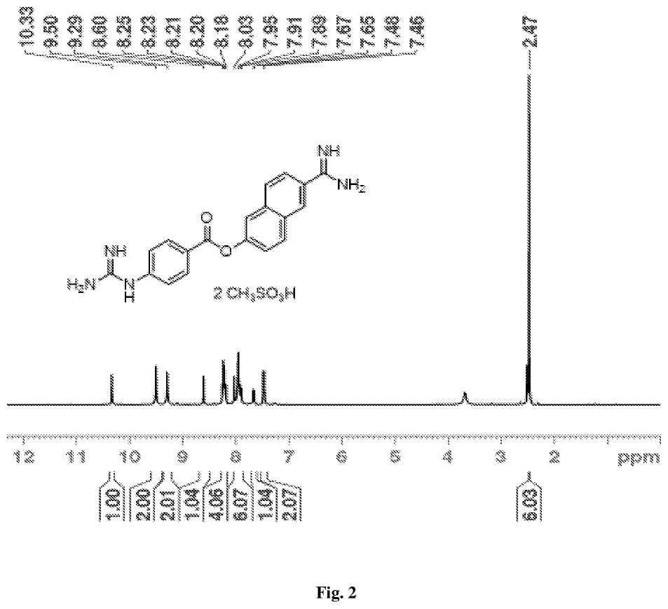

FIG. 3 depicts ¹³C-NMR of Nafamaostat mesylate.

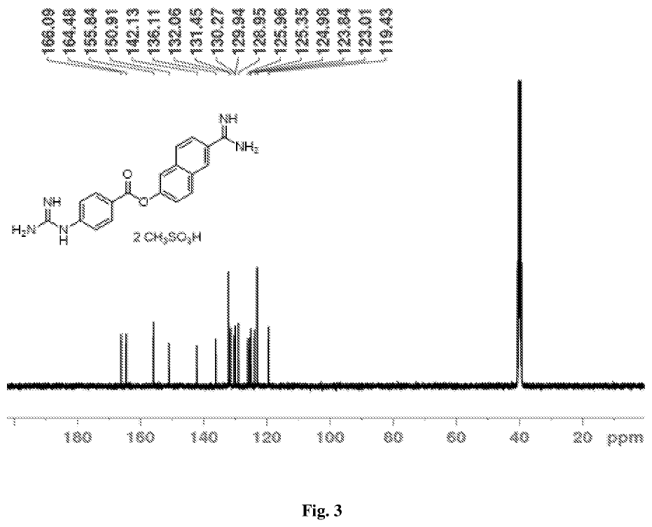

FIG. 4 depicts DEPT of Nafamaostat mesylate.

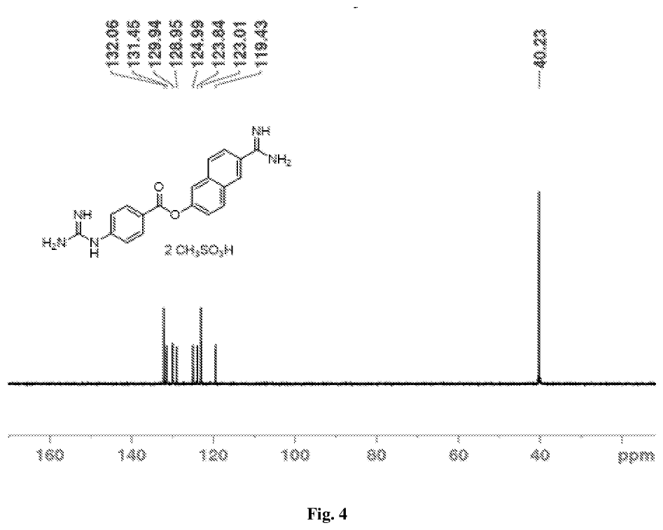

FIG. 5 depicts ¹H-NMR of Camostat mesylate.

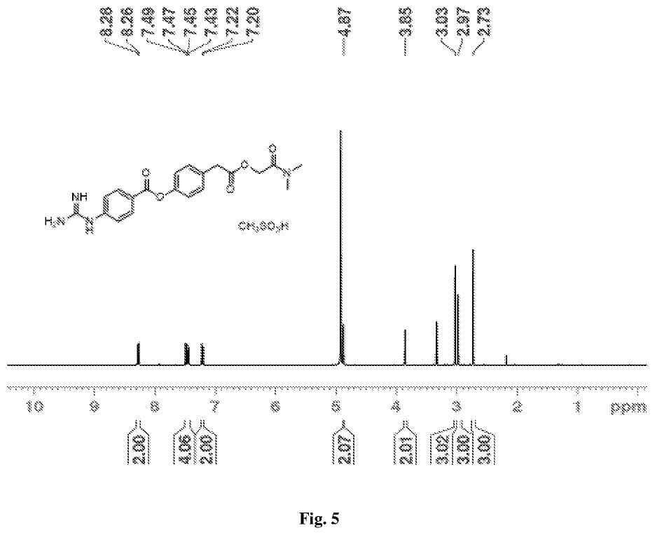

FIG. 6 depicts ¹³C-NMR of Camostat mesylate.

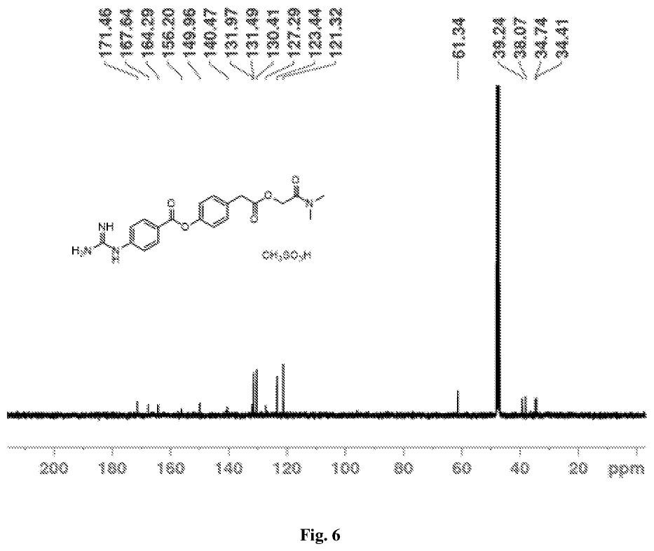

FIG. 7 depicts DEPT of Camostat mesylate.

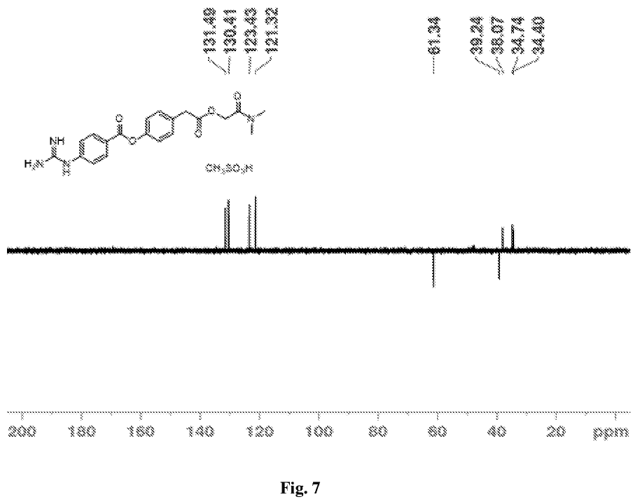

### DETAILED DESCRIPTION OF THE INVENTION

The main aspect of the present invention is to provide a process for compound of general Formula (F) comprising of;

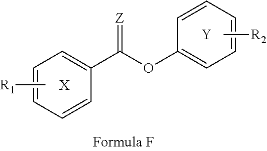

wherein Z is O, S, NH;

Ring X and Y is aryl, heteroaryl, extended rings selected from the group consisting of naphthalene, phenanthrene, quinoline, isoquinoline;

R₁ is guanidinyl, amidinyl, 2-(dimethylamino)-2-oxoethyl acetyl, halogens, alkyl groups, amide group, cyano group, nitro group, amino group, methoxy group, O-benzyl esters, N-benzyl esters, hydroxyl group, aryl groups, heteroaryl groups;

R₂ is guanidinyl, amidinyl, 2-(dimethylamino)-2-oxoethyl acetyl, halogens, alkyl groups, amide group, cyano group, nitro group, amino group, methoxy group, O-benzyl esters, N-benzyl esters, hydroxyl group, aryl groups, heteroaryl groups, substituted benzenes;
i. reacting 2,4,6-trihalo-1,3,5-triazine and base at 25-40° C. for 5 minutes to obtain activated complex;
ii. reacting the activated complex as obtained in step-i with fragment A at 40-60° C. for 4 hours to obtain intermediate complex;
iii. reacting the intermediate compound obtained in step-ii with fragment B in the ratio of ranging from 0-1 and with fragment C in the ratio of ranging from 0-1 to obtain compound D;
iv. treating the compound D as obtained in step-iii with aqueous sodium bicarbonate;
v. optionally purifying the obtained bicarbonate salt of compound D as obtained in step-iv;
vi. treating the compound D as obtained in step iv & v with methanesulfonic acid in presence of methanol to provide Formula F.

Within the context of this embodiment the coupling agent trihalotriazine is selected from trichlorotriazine, tribromotriazine, trifluorotriazine, and more preferably inexpensive trichlorotriazine & base is selected from pyridine (py), N-methylmorpholine (NMM) triethylamine (Et₃N) Diazabicycloundecene (DBU), more preferably N-methylmorpholine (NMM).

In an embodiment of present invention, the fragment A is prepared by reacting p-aminobenzoic acid (1) with thiourea (4) in presence of base in ethanol.

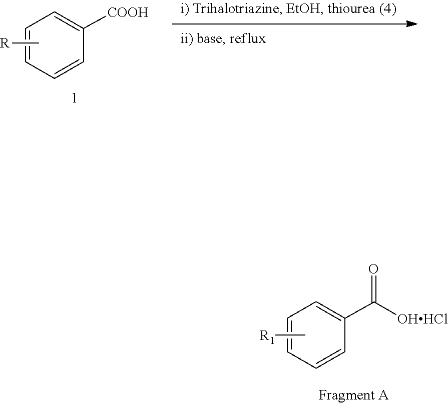

Within the context of this embodiment base is selected from the list of pyridine (py), N-methylmorpholine (NMM) triethylamine (Et₃N) Diazabicycloundecene (DBU), more preferably N-methylmorpholine (NMM).

In accordance with another embodiment, the present invention provides a process for the preparation of Nafamostat by the coupling of fragment A1 namely p-guanidinobenzoic acid and fragment B1 namely 6-hydroxy-2-naphthimidamide using trichlorotriazine, tribromotriazine, trifluorotriazine, and more preferably inexpensive trichlorotriazine (TCT) as coupling reagent in the presence of base such as pyridine (py), N-methylmorpholine (NMM) triethylamine (Et₃N) Diazabicycloundecene (DBU) and more preferably N-methylmorpholine (NMM). The Nafamostat (D1) was precipitated as carbonate salt after treating with bicarbonate salt, following by acidification with methanesulfonic acid (MSA) to obtained Nafamostat mesylate (F).

In accordance with another embodiment, the present invention provides a process for the preparation of Fragment A1 by the reaction of p-aminobenzoic acid 1 with thioureatrioxide 2 in presence of base selected from sodium carbonate, cesium carbonate, ammonium carbonate and more preferably potassium carbonate.

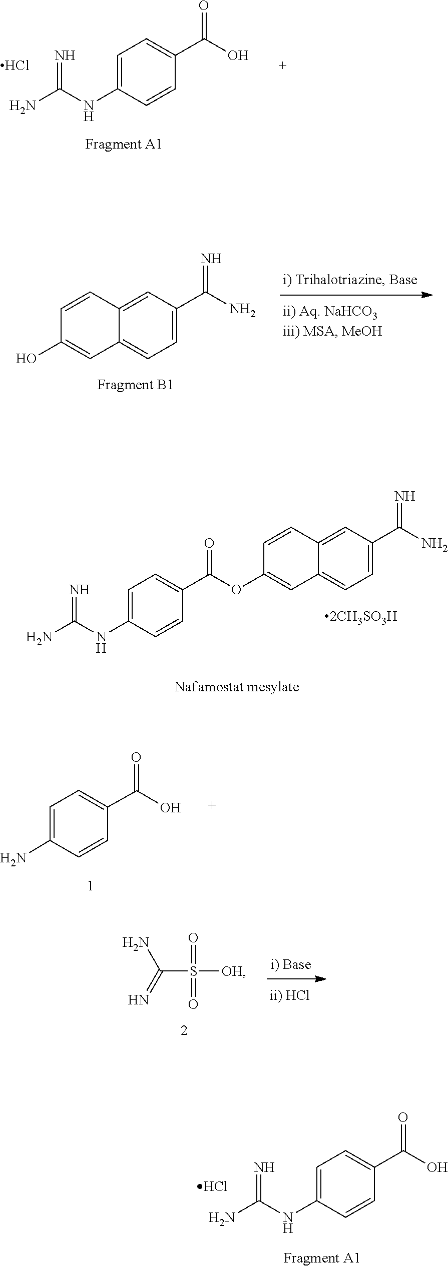

In accordance with another embodiment, the present invention provides a process for the synthesis of thioureatrioxide 2 from thiourea 4 by oxidation using hydrogen peroxide/peracetic acid system.

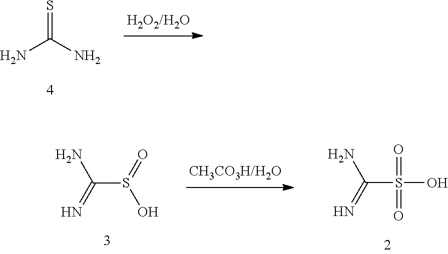

In accordance with another embodiment, the present invention provides a process for the synthesis of fragment B1 from compound 5 via acid catalysed conversion of cyano to amidine.

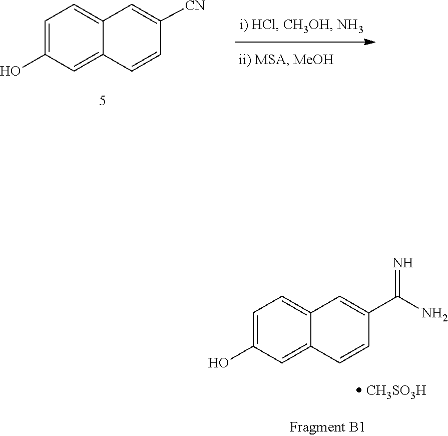

In accordance with another embodiment, the present invention provides a process for the synthesis of compound 5 from compound 6 via conversion of bromo to cyano group.

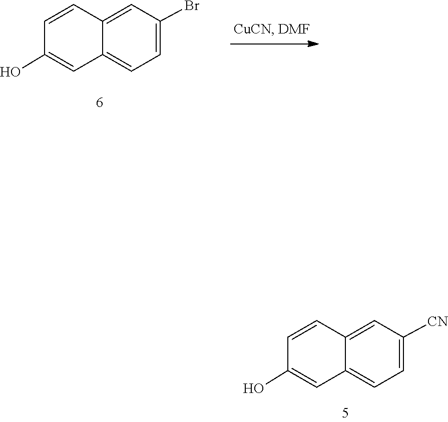 [SMILES: Oc1ccc2cc(Br)ccc2c1 ; N#Cc1ccc2cc(O)ccc2c1]

In accordance with another embodiment, the present invention provides a process for the synthesis of compound 6 from β-napthol 7 via two step reactions.

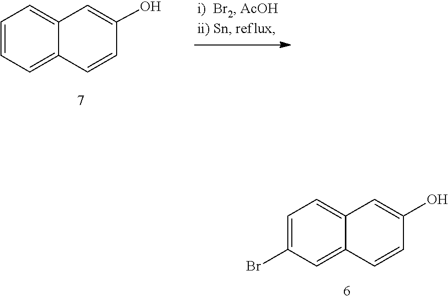 [SMILES: Oc1ccc2ccccc2c1 ; Oc1ccc2cc(Br)ccc2c1]

In accordance with another embodiment, the present invention provides a process for the preparation of Camostat by the coupling of fragment A1 namely p-guanidinobenzoic acid and fragment C1 namely 2-(dimethylamino)-2-oxoethyl 2-(4-hydroxyphenyl) acetate using trichlorotriazine, tribromotriazine, trifluorotriazine, and more preferably inexpensive trichlorotriazine as coupling reagent in the presence of base such as pyridine (py), N-methylmorpholine (NMM) triethylamine (Et₃N) Diazabicycloundecene (DBU), more preferably N-methylmorpholine (NMM). The Camostat (D2) was precipitated as carbonate salt after treating with bicarbonate salt, following by acidification with methanesulfonic acid (MSA) to obtained Camostat mesylate (F).

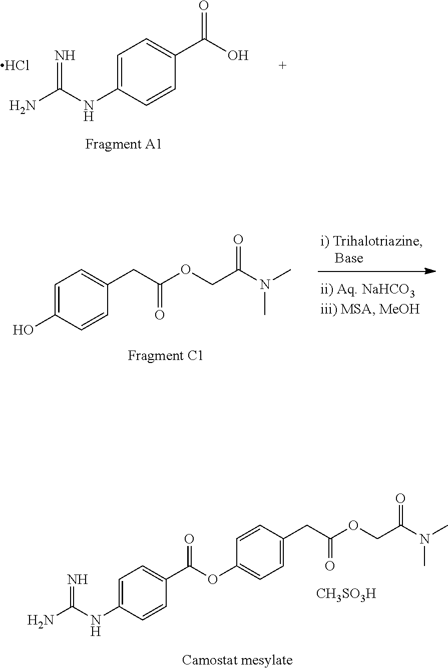

In accordance with another embodiment, the present invention provides a process for the synthesis of fragment C1 by the coupling of 4-hydroxyphenyl acetic acid 8 with N,N-dimethylbromoacetamide 9.

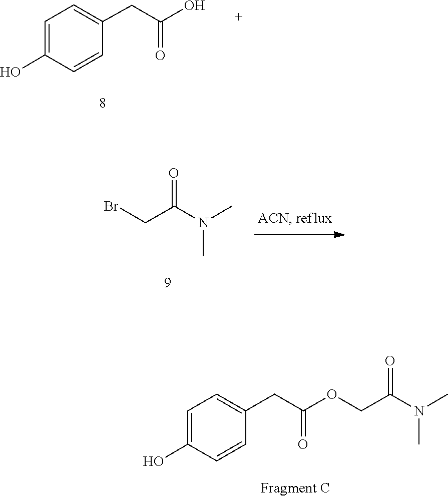

In accordance with another embodiment, the present invention provides a process for the synthesis of compound 9 from the coupling of bromoacetylbromide 10 with N,N-dimethylamine hydrochloride.

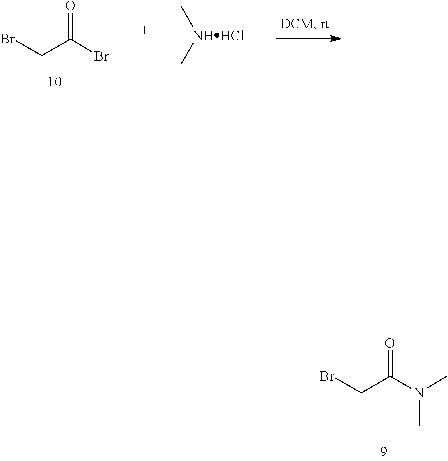

In accordance with another embodiment, the present invention provides a process for the synthesis of fragment A from the coupling of p-aminobenzoic acid 1 with thiourea 4 using trichlorotriazine, tribromotriazine, trifluorotriazine, and more preferably inexpensive trichlorotriazine as coupling reagent.

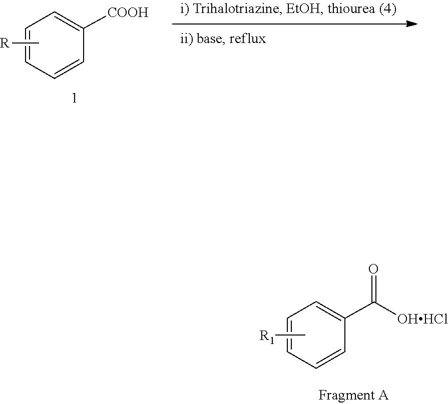

All the product mixtures were analysed by thin layer chromatography. UV inactive compounds were visualized in staining solution and UV active compounds were detected with UV lamp (λ=254 nm). All the reactions were performed under inert atmosphere wherever required.

NMR spectra (¹HNMR, ¹³C, DEPT) were recorded in 400 MHz spectrometer using CDCl₃ and CD₃OD solvent.

ES1-MS and HRMS spectra were recorded on LC-MS/MS and HRMS-6540-UHD machines. Optical rotations were measured on a Perkin Elmer polarimeter.

Column chromatography was carried out with silica gel (60-120, 230-400 mesh).

Trichlorotriazine (TCT) as coupling reagent being inexpensive and stable and its use provides cost effective route for the synthesis of Nafamostat, Camostat and their derivatives.

Consumption of TCT as a coupling reagent make the preparation of Nafamostat or Camostat simple, high yielding, low impurities and suitable for industrial production.

Scheme for the synthesis of fragment A1, B1, C1 and Nafamostat mesylate and Camostat mesylate are provided in examples. Table 1 and Table 2 provide the reactants used and the reaction conditions with resulting products and yield thereof.

TABLE 1

| | | | | Reactants | |
|---|---|---|---|---|---|
| | | | | (TCT, Base; | |
| | | | | Aq | |
| | | | Conditions of | NaHCO₃; | |
| | Reactant | Reactant | pressure temp | MSA, | Products |
| Entry | (Fragment A₁) | (Fragment B₁) | etc | MeOH) | (Formula F) |
| 1. | 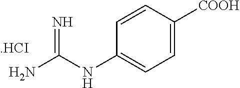 [SMILES: ".HCI" ; N=C(N)Nc1ccc(C(=O)O)cc1] | 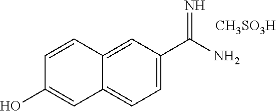 | Reaction was stirred at room temperature for 5 min., then at 60° C. for 4 hours and overnight at room temperature. | TCT: 5 g, 26.88 mmol, NMM: 70 mL, AqNaHCO: 70 ml, MSA: 2.5 mL, MeOH: 20 ml. | 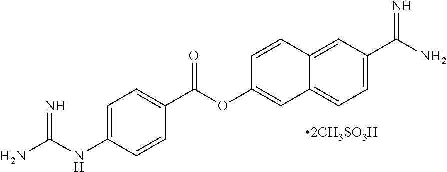 |
| | p-guanidinobenzoic | 6-hydroxy-2- | | | Nafamostat |
| | acid (9.62 g, | naphthimidamide | | | mesylate (13 g, |
| | 53.76 mmol) | (10 g, 53.76 | | | yield 70 %) |
| | | mmol) | | | |
| 2. | 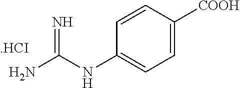 [SMILES: ".HCI" ; N=C(N)Nc1ccc(C(=O)O)cc1] | 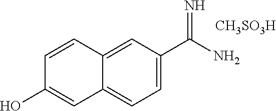 | Reaction was stirred at room temperature for 5 min., then at 60° C. for 4 hours and overnight at room temperature. | TCT: 5 g, 26.88 mmol, Pyridine: 70 mL, AqNaHCO: 70 ml, MSA: 2.5 mL, MeOH: 20 ml. | 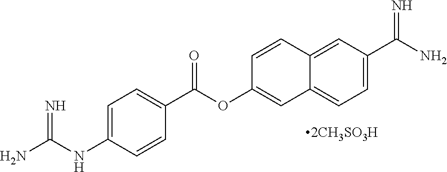 |
| | p- | 6-hydroxy-2- | | | Nafamostat |
| | guanidinobenzoic | naphthimidamide | | | mesylate (11.7 g, |
| | acid (9.62 g, | (10 g, 53.76 | | | yield 63 %) |
| | 53.76 mmol) | mmol) | | | |
| 3. | 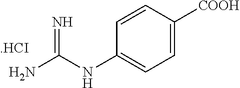 [SMILES: ".HCI" ; N=C(N)Nc1ccc(C(=O)O)cc1] | 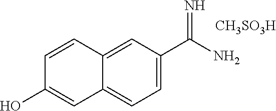 | Reaction was stirred at room temperature for 5 min., then at 60º C for 4 hours and overnight at room temperature. | TCT: 5 g, 26.88 mmol, Et₃N: 70 mL, AqNaHCO: 70 ml, MSA: 2.5 mL, MeOH: 20 ml. | 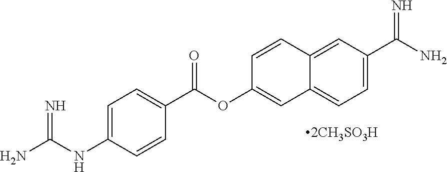 |
| | p- | 6-hydroxy-2- | | | Nafamostat |
| | guanidinobenzoic | naphthimidamide | | | mesylate (11 g, |
| | acid (9.62 g, | (10 g, 53.76 | | | yield 60 %) |
| | 53.76 mmol) | mmol) | | | |
| 4. | 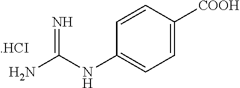 [SMILES: ".HCI" ; N=C(N)Nc1ccc(C(=O)O)cc1] | 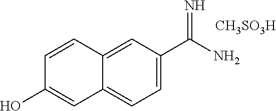 | Reaction was stirred at room temperature for 5 min., then at 60° C. for 4 hours and overnight at room temperature. | TCT: 5 g, 26.88 mmol, DBU: 70 mL AqNaHCO: 70 ml, MSA: 2.5 mL, MeOH: 20 ml. | 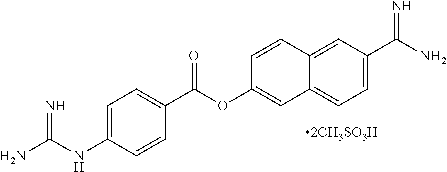 |
| | p- | 6-hydroxy-2- | | | Nafamostat |
| | guanidinobenzoicacid | naphthimidamide | | | mesylate (10 g, |
| | (9.62 g, | (10 g, 53.76 | | | yield 54 %) |
| | 53.76 mmol) | mmol) | | | |

TABLE 2

| | | | | Reactants | |
|---|---|---|---|---|---|
| | | | | (TCT, | |
| | | | | Base; Aq | |
| | Reactant | | | NaHCO₃; | |
| | (Fragment | Reactant | Conditions of | MSA, | Products |
| Entry | A₁) | (Fragment C₁) | pressure temp etc | MeOH) | (Formula F) |
| 1. | 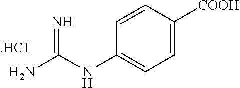 [SMILES: ".HCI" ; N=C(N)Nc1ccc(C(=O)O)cc1] | 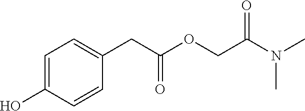 [SMILES: CN(C)C(=O)COC(=O)Cc1ccc(O)cc1] | Reaction was stirred at room temperature for 5 min., then at 60° C. for 4 hours and overnight at room temperature. | TCT: 0.250 g, 1.39 mmol, NMM: 20 mL, | 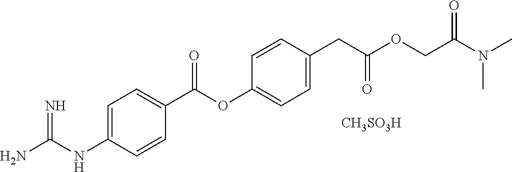 |
| | p-guanidinobenzoic | 2-(dimethylamino)- | | | Camostat |
| | acid | 2-oxoethyl 2-(4- | | | mesylate |
| | (0.500 g, 2.79 | hydroxyphenyl) | | | (0.75 g, yield |
| | mmol) | acetate (0.662 g, | | | 28 %) |
| | | 2.79 mmol) | | | |
| 2. | 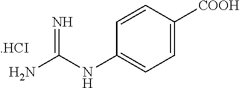 [SMILES: ".HCI" ; N=C(N)Nc1ccc(C(=O)O)cc1] | 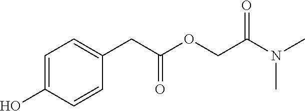 [SMILES: CN(C)C(=O)COC(=O)Cc1ccc(O)cc1] | Reaction was stirred at room temperature for 5 min., then at 60° C. for 4 hours and overnight at room temperature. | TCT: 0.250 g, 1.39 mmol, Pyridine: 20 mL, | 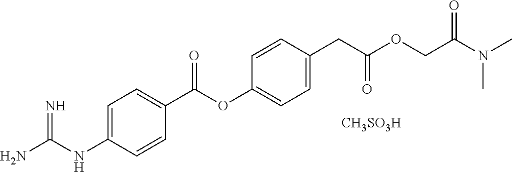 |
| | p-guanidinobenzoic | 2-(dimethylamino)- | | | Camostat |
| | acid | 2-oxoethyl 2-(4- | | | mesylate |
| | (0.500 g, 2.79 | hydroxyphenyl) | | | (0.20 g, yield 23%) |
| | mmol) | acetate (0.662 g, | | | |
| | | 2.79 mmol) | | | |
| 3. | 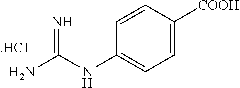 [SMILES: ".HCI" ; N=C(N)Nc1ccc(C(=O)O)cc1] | 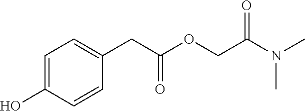 [SMILES: CN(C)C(=O)COC(=O)Cc1ccc(O)cc1] | Reaction was stirred at room temperature for 5 min., then at 60° C. for 4 hours and overnight at room temperature. | TCT: 0.250 g, 1.39 mmol, Et₃N: 20 mL, | 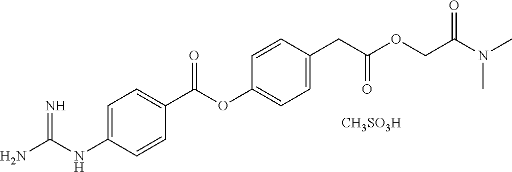 |
| | p-guanidinobenzoic | 2-(dimethylamino)- | | | Camostat |
| | acid | 2-oxoethyl 2-(4- | | | mesylate |
| | (0.500 g, 2.79 | hydroxyphenyl) | | | (0.17 g, yield |
| | mmol) | acetate (0.662 g, | | | 20 %) |
| | | 2.79 mmol) | | | |
| 4. | 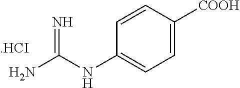 [SMILES: ".HCI" ; N=C(N)Nc1ccc(C(=O)O)cc1] | 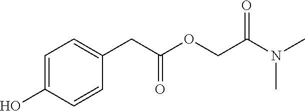 [SMILES: CN(C)C(=O)COC(=O)Cc1ccc(O)cc1] | Reaction was stirred at room temperature for 5 min., then at 60° C. for 4 hours and overnight at room temperature. | TCT: 0.250 g, 1.39 mmol, DBU: 20 mL, | 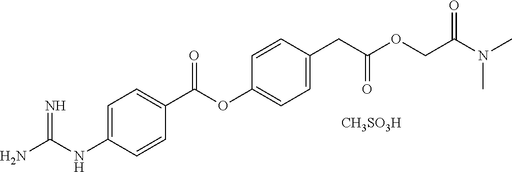 |
| | p-guanidinobenzoic acid | 2-(dimethylamino)- | | | Camostat |
| | (0.500 g, 2.79 | 2-oxoethyl 2-(4- | | | mesylate |
| | mmol) | hydroxyphenyl)a | | | (0.15 g, yield |
| | | cetate (0.662 g, | | | 17 %) |
| | | 2.79 mmol) | | | |

### EXAMPLES

The following examples are given by way of illustration and therefore should not be construed to limit the scope of the present invention.

### Example 1

### Synthesis of Fragment A1 (p-Guanidinobenzoic Acid Hydrochloride)

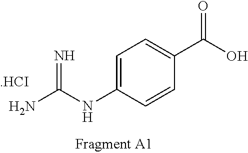 [SMILES: ".HCI" ; N=C(N)Nc1ccc(C(=O)O)cc1]

Step 1: Synthesis of thiourea dioxide (3) and thiourea trioxide (2):

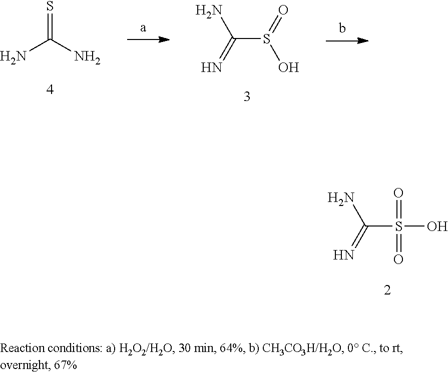

50 g of thiourea (4) (0.657 mol, 1 eq.) was added into 200 mL of hot water at 40° C. and thoroughly stirred to allow thiourea to dissolve completely. The aqueous thiourea solution thus prepared was cooled and then 371.42 mL of hydrogen peroxide (concentration: 30%) (4.6 mol, 7 eq.) was added slowly at a rate such that the solution temperature was held below 10° C. Thereafter, the solution was cooled to 0° C. and stirred for about 30 minutes to allow crystals to be aged. After the crystal ageing, the solid-liquid mixture at 0° C. was immediately filtered and the fractionated crystals were dried at 50° C. Further, washing of the reaction mixture has been done with ethanol to expel out the unreacted thiourea. The yield of the crystals thus obtained was 64% to yield 45 g of thiourea dioxide (3) respectively. Peracetic acid (48.54 mL, 0.664 mol) was stirred at 0° C. for 0.5 h followed by addition of thiourea dioxide (3) (18 g, 0.664 mol) in water (100 mL), which was slowly warmed to room temperature and stirred overnight. The precipitate (2) (thiourea trioxide) was removed by filtration and the resulted solution was evaporated on rotavapor and further washed with ethanol to obtain the thiourea trioxide (2) (17 g, yield: 67%) respectively m.p.=132-134° C., MS (ESI+) m/z calcd. for CH₅N₂O₃S, 125.01 (M+H); found 125.12.

Step 2: Synthesis of 4-guanidinobenzoic Acid (A1)

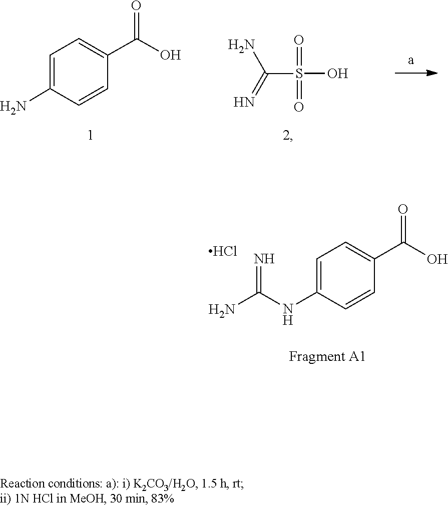 [SMILES: Nc1ccc(C(=O)O)cc1 ; N=C(N)S(=O)(=O)O ; Cl ; N=C(N)Nc1ccc(C(=O)O)cc1]

Potassium carbonate (6.01 g, 0.0435 mol) was added to 4-aminobenzoic acid (1) (6 g, 0.0435 mol) in water (40 ml). The mixture was stirred for 0.5 h at room temperature and then thiourea trioxide (2) (8.14 g, 0.0653 mol) was added slowly. White solid was precipitated and the mixture was stirred at room temperature for another 1 h. After filtration, the white solid was dissolved in the solution of 1 N hydrogen chloride in methanol (50 mL) and stirred for 0.5 h. The solvent was removed on rotavapor and the product was obtained as its hydrochloride (6.5 g, yield: 83%), m.p.=280-285° C. ¹H NMR (400 MHz, DMSO-d⁶) δ 11.16 (s, 1H), 10.19 (s, 1H), 7.97-7.99 (d, J=8 Hz, 2H), 7.70 (s, 3H), 7.33-7.35 (d, J=8 Hz, 2H), MS (ESI+) m/z calcd for C₈H₁₀N₃O₂, 180.07 (M+H); found 180.18.

### Example 2

### Synthesis of Fragment B1 (6-hydroxy-2-naphthimidamide methane sulfonate)

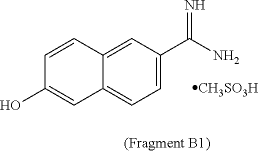

Step 1: Synthesis of 6-bromonaphthalen-2-ol (6):

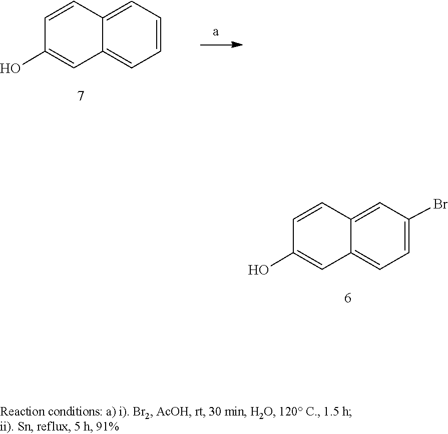 [SMILES: Oc1ccc2ccccc2c1 ; Oc1ccc2cc(Br)ccc2c1]

β-naphthol (7) 40 g (1 mole) and 100 ml of glacial acetic acid were taken in a round-bottom flask fitted with a dropping funnel and a reflux condenser. A solution of 88 g (2 mole) of bromine in 30 ml of acetic acid was added through the dropping funnel for a period of 15-30 minutes. The flask was shaken gently during the addition, β-naphthol dissolves during this period, and heat was evolved; the mixture was cooled somewhat towards the end of the addition to avoid excessive loss of hydrogen bromide, 30 ml of water was then added, and the mixture was heated to boiling. The reaction mixture was then cooled to 100° C., 5 g of mossy tin is added and boiled until the metal is dissolved. A second portion of 5 g of tin was then added and dissolved by boiling, and finally a third portion of 32 g (a total of 42 g) of tin was introduced. The mixture was boiled for 3 h, cooled to 50° C., and filtered with suction. The crystalline tin salts which were thus removed are washed on the funnel with 100 ml of cold acetic acid, the washings being added to the main portion of the filtrate. This filtrate was stirred in cold water; the 6-bromo-2-naphthol (6) which was precipitated was filtered with suction, removed from the funnel, and washed by stirring with 100 ml of cold water. After filtering again and drying at 100° C. to obtain 6-bromo-2-naphthol (6) 60 g (yield 91%). This crude product melts at 123-127° C. contains some tin but is pure enough for most purposes. ¹H NMR (400 MHz, CDCl₃) δ 10.08 (s, 1H), 7.96 (s, 1H), 7.72-7.70 (d, 1H, J=8 Hz), 7.62-7.60 (d, 1H, J=8 Hz), 7.45-7.42 (m, 1H), 7.13-7.11 (m, 2H), MS (ESI+) m/z calcd for C₁₀H₈BrO, 222.97 (M+H); found 224.08.

Step 2: Synthesis of 6-hydroxy-2-naphthonitrile (5):

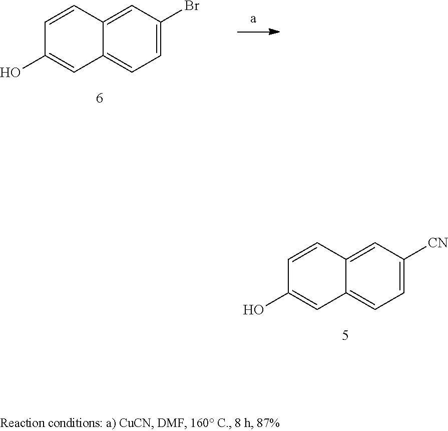 [SMILES: Oc1ccc2cc(Br)ccc2c1 ; N#Cc1ccc2cc(O)ccc2c1]

A mixture of 30 g of 6-bromo-2-naphthol (6) and 15 g of CuCN in DMF was vigorously stirred at 150° C. for 5-6 h. After cooling to room temperature, the mixture was added 10% NaOH and stirred for 5 min. The reaction was filtered and the filtrate was washed with water. Then, 3 N HCl was used to adjust pH to 2-3. The precipitate was collected by filtration to give 6-hydroxy-2-naphthonitrile (5) as a brown solid (20 g, 87%) m.p.=165-170° C. ¹H NMR (400 MHz, CDCl₃) δ 10.05 (s, 1H), 8.50 (s, 1H), 8.07-8.05 (d, 1H, J=8 Hz), 7.95 (m, 1H) 7.50-7.45 (m, 2H), 7.04-7.02 (m, 1H), MS (ESI+) m/z calcd for C₁₁H₈NO, 170.06 (M+H); found 170.19.

Step 3: Synthesis of 6-hydroxy-2-naphthimidamide methane sulfonate (B1):

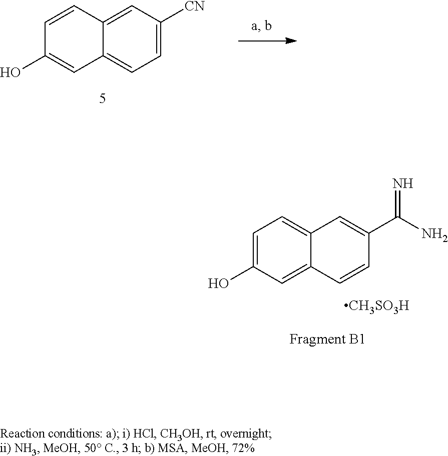

10 g (50 mmol) of 6-hydroxy-2-naphthonitrile (5) was added to a cooled saturated MeOH—HCl solution (50 ml) and the mixture was stirred overnight at room temperature and concentrated. The residue was dissolved in MeOH (50 ml) and gaseous NH₃ was introduced into the solution at 50° C. for 3 h. The mixture was concentrated in vacuo and saturated NaHCO3 solution was added to the residue with stirring. The precipitate was collected, washed with water and then with acetone, and a suspension of the precipitate in MeOH (10 ml) was treated with MSA (5.8 g, 60 mmol). Et₂O was added to the solution and the precipitate was collected to give 6-hydroxy-2-naphthimidamide methane sulfonate (B1) (9.2 g, 65%). Re-crystallization from EtOH afforded an analytical sample as a pale yellow powder: mp 227-228° C. ¹H NMR (400 MHz, CDCl₃) δ 9.20 (s, 2H), 8.95 (s, 2H), 8.37 (s, 1H), 7.92-7.90 (d, J=8 Hz 1H), 7.85-7.83 (d, J=8 Hz, 1H), 7.72-7.70 (d, J=8 Hz, 1H), 7.23(s, 2H), 2.49 (s,1H), 2.45 (s, 3H), MS (ESI+) m/z calcd for C₁₁H₁₁N₂O, 187.08 (M+H); found 187.22.

### Example 3

### Synthesis of Fragment C1 (2-(dimethylamino)-2-oxoethyl 2-(4-hydroxyphenyl)acetate)

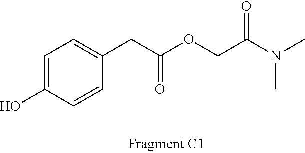 [SMILES: CN(C)C(=O)COC(=O)Cc1ccc(O)cc1]

Step 1: Synthesis of 2-bromo-N,N-dimethylacetamide (9).

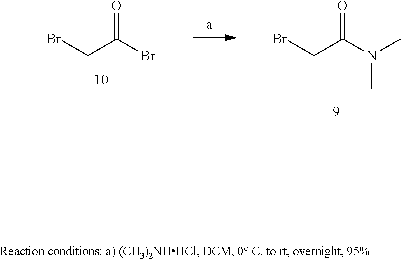

Bromoacetyl bromide (10) (1.52 mL, 17.28 mmol) was dissolved in CH₂Cl₂ (20 mL), and dimethylammonium chloride (1 g, 12.34 mmol) was added at 0° C. The reaction mixture was stirred overnight at rt. The reaction mixture was then subjected to work up with DCM and water. The organic layer was dried (Na₂SO₄) and concentrated in vacuum. The crude product (9) was obtained as a yellow oil (0.450 g, 54%) and can be used further without purification. ¹H NMR (400 MHz, DMSO-d⁶) δ 4.34 (s, 2H), 3.04 (s, 3H), 2.89 (s, 3H), m/z=164.97, MS (ESI+) m/z calcd for C₄H₉BrNO, 165.98 (M+H); found 167.02.

Step 2: Synthesis of 2-(dimethylamino)-2-oxoethyl 2-(4-hydroxyphenyl) acetate (C1):

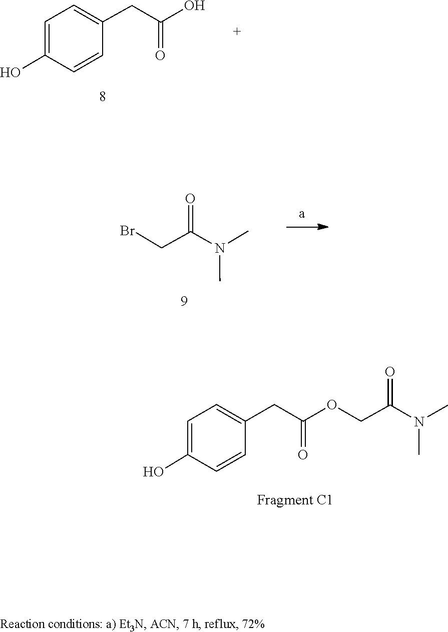

2-Bromo-N,N-dimethylacetamide (9) (0.50 g, 3.01 mmol) and p-hydroxyphenylacetic acid (8) (0.450 g, 3.01 mmol) were dissolved in 10 ml of acetonitrile, and triethylamine (0.425 g, 4.21 mmol) was added to the solution. The resulting mixture was refluxed for 7 h. Upon completion of reaction (checked by TLC), solvent was evaporated and fragment C1 was purified by column chromatography. TLC (Hexane/EtOAc, 2:8) R_{f}=0.4; Yield (0.60 g, 81%); white solid; m.p.: 103-107° C. ¹H NMR (400 MHz, DMSO-d⁶) δ 9.39 (s, 1H), 7.10-7.08 (d, J=8 Hz, 2H), 6.74-6.72 (d, J=8 Hz, 2H), 4.75 (s, 2H), 3.61 (s, 2H), 2.85 (s, 3H), 2.79 (s, 3H), MS (ESI+) m/z calcd for C₁₂H₁₆NO₄ 238.10 (M+H); found 238.26.

### Example 4

### Synthesis of 6-carbamimidoylnaphthalen-2-yl 4-guanidinobenzoate dimesylate (Nafamostat mesylate)

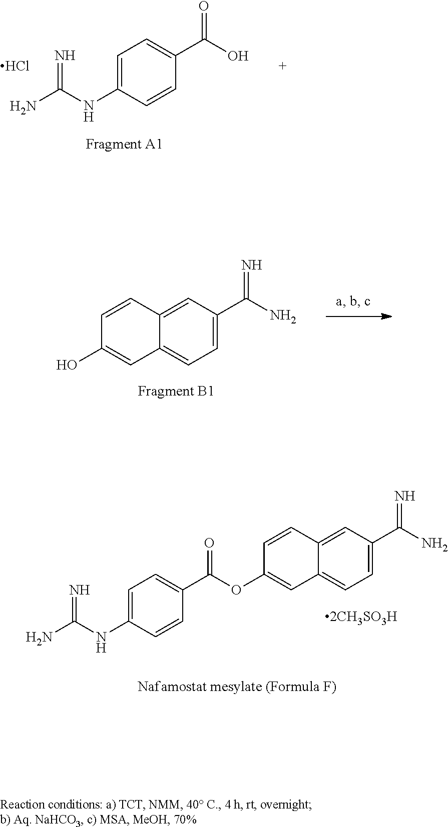 [SMILES: Cl ; N=C(N)Nc1ccc(C(=O)O)cc1 ; N=C(N)c1ccc2cc(O)ccc2c1 ; N=C(N)Nc1ccc(C(=O)Oc2ccc3cc(C(=N)N)ccc3c2)cc1]

2,4,6-trichloro-1,3,5-triazine (5 g, 26.88 mmol) and 4-methylmorpholine (70 ml) were taken in a round-bottom flask and stirred at room temperature for 5 minutes. To the reaction mixture 4-guanidinobenzoic acid, fragment A1 (9.62 g, 53.76 mmol) was added and stirred at 40° C. for 4 hours. Then 6-hydroxy-2-naphthimidamide, fragment B1 (10 g, 53.76 mmol) was added and the reaction mixture was stirred overnight at room temperature. Upon completion of the reaction i.e, formation of product D1 (monitored by TLC), bicarbonate solution was added to the reaction mixture and stirred for 10 minutes for the precipitation of the product. The precipitates were collected by filtration and subsequently washed with water, acetone and methanol. The precipitates were then dissolved in methanol and treated with methanesulfonic acid. On adding diethyl ether Nafamostat mesylate (F) precipitate out (13 g, yield 70%) m.p.=257-262° C. ¹H NMR (400 MHz, DMSO-d⁶) δ 10.33 (s, 1H), 9.50 (s, 2H), 9.29 (s, 2H), 8.60 (s, 1H), 8.25-8.17 (m, 4H), 8.02-7.88 (m, 6H), 7.66-7.64 (d, J=8 Hz, 1H), 7.47-7.45 (d, J=8 Hz, 2H), 2.47 (s, 6H). ¹³C NMR (101 MHz, DMSO-d⁶) δ 166.0, 164.4, 155.8, 150.9, 142.1, 136.1, 132.0, 131.4, 130.2, 129.9, 128.9, 125.9, 125.3, 124.9, 123.8, 123.0, 119.4, MS (ESI+) m/z calcd for C₁₉H₁₈N₅O₂ 348.14 (M+H); found 348.38.

### Example 5

### Synthesis of 6-carbamimidoylnaphthalen-2-yl 4-guanidinobenzoate dimesylate (Nafamostat mesylate)

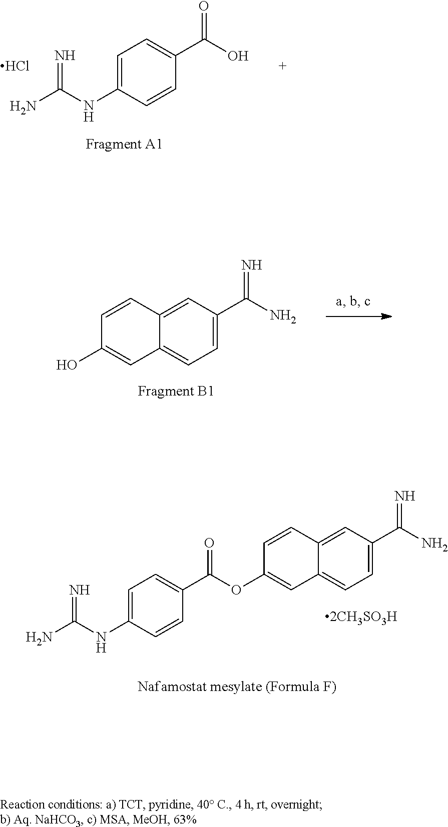 [SMILES: Cl ; N=C(N)Nc1ccc(C(=O)O)cc1 ; N=C(N)c1ccc2cc(O)ccc2c1 ; N=C(N)Nc1ccc(C(=O)Oc2ccc3cc(C(=N)N)ccc3c2)cc1]

2,4,6-trichloro-1,3,5-triazine (5 g, 26.88 mmol) and 4-methylmorpholine (70 ml) were taken in a round-bottom flask and stirred at room temperature for 5 minutes. To the reaction mixture 4-guanidinobenzoic acid, fragment A1 (9.62 g, 53.76 mmol) was added and stirred at 40° C. for 4 hours. Then 6-hydroxy-2-naphthimidamide, fragment B1 (10 g, 53.76 mmol) was added and the reaction mixture was stirred overnight at room temperature. Upon completion of the reaction i.e, formation of product D1 (monitored by TLC), bicarbonate solution was added to the reaction mixture and stirred for 10 minutes for the precipitation of the product. The precipitates were collected by filtration and subsequently washed with water, acetone and methanol. The precipitates were then dissolved in methanol and treated with methanesulfonic acid. On adding diethyl ether Nafamostat mesylate (F) precipitate out (11.7 g, yield 63%).

### Example 6

### Synthesis of 6-carbamimidoylnaphthalen-2-yl 4-guanidinobenzoate dimesylate (Nafamostat mesylate)

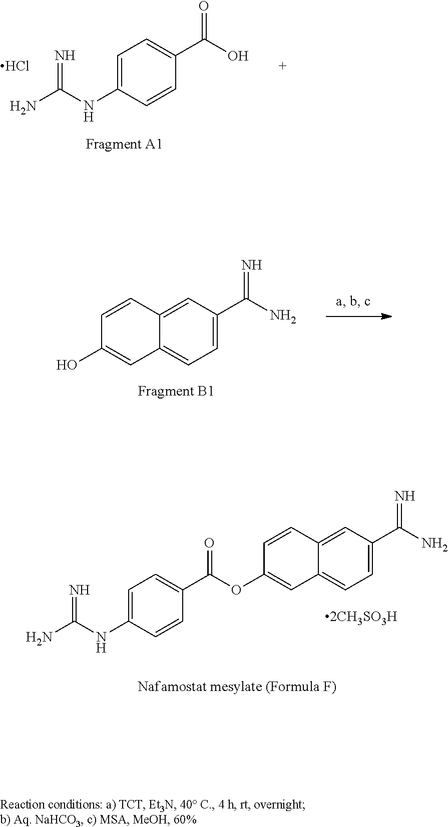 [SMILES: Cl ; N=C(N)Nc1ccc(C(=O)O)cc1 ; N=C(N)c1ccc2cc(O)ccc2c1 ; N=C(N)Nc1ccc(C(=O)Oc2ccc3cc(C(=N)N)ccc3c2)cc1]

2,4,6-trichloro-1,3,5-triazine (5 g, 26.88 mmol) and 4-methylmorpholine (70 ml) were taken in a round-bottom flask and stirred at room temperature for 5 minutes. To the reaction mixture 4-guanidinobenzoic acid, fragment A1 (9.62 g, 53.76 mmol) was added and stirred at 40° C. for 4 hours. Then 6-hydroxy-2-naphthimidamide, fragment B1 (10 g, 53.76 mmol) was added and the reaction mixture was stirred overnight at room temperature. Upon completion of the reaction i.e, formation of product D1 (monitored by TLC), bicarbonate solution was added to the reaction mixture and stirred for 10 minutes for the precipitation of the product. The precipitates were collected by filtration and subsequently washed with water, acetone and methanol. The precipitates were then dissolved in methanol and treated with methanesulfonic acid. On adding diethyl ether Nafamostat mesylate (F) precipitate out (11 g, yield 60%).

### Example 7

### Synthesis of 6-carbamimidoylnaphthalen-2-yl 4-guanidinobenzoate dimesylate (Nafamostat mesylate)

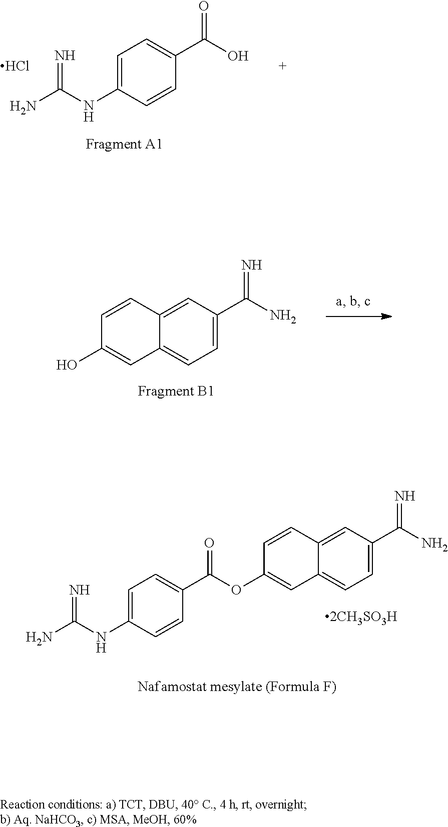 [SMILES: Cl ; N=C(N)Nc1ccc(C(=O)O)cc1 ; N=C(N)c1ccc2cc(O)ccc2c1 ; N=C(N)Nc1ccc(C(=O)Oc2ccc3cc(C(=N)N)ccc3c2)cc1]

2,4,6-trichloro-1,3,5-triazine (5 g, 26.88 mmol) and 4-methylmorpholine (70 ml) were taken in a round-bottom flask and stirred at room temperature for 5 minutes. To the reaction mixture 4-guanidinobenzoic acid, fragment A1 (9.62 g, 53.76 mmol) was added and stirred at 40° C. for 4 hours. Then 6-hydroxy-2-naphthimidamide, fragment B1 (10 g, 53.76 mmol) was added and the reaction mixture was stirred overnight at room temperature. Upon completion of the reaction i.e, formation of product D1 (monitored by TLC), bicarbonate solution was added to the reaction mixture and stirred for 10 minutes for the precipitation of the product. The precipitates were collected by filtration and subsequently washed with water, acetone and methanol. The precipitates were then dissolved in methanol and treated with methanesulfonic acid. On adding diethyl ether Nafamostat mesylate (F) precipitate out (10 g, yield 54%).

### Example 8

### Synthesis of 4-(2-(2-(dimethylamino)-2-oxoethoxy)-2-oxoethyl)phenyl 4-guanidinobenzoate methanesulfonate (Camostat mesylate)

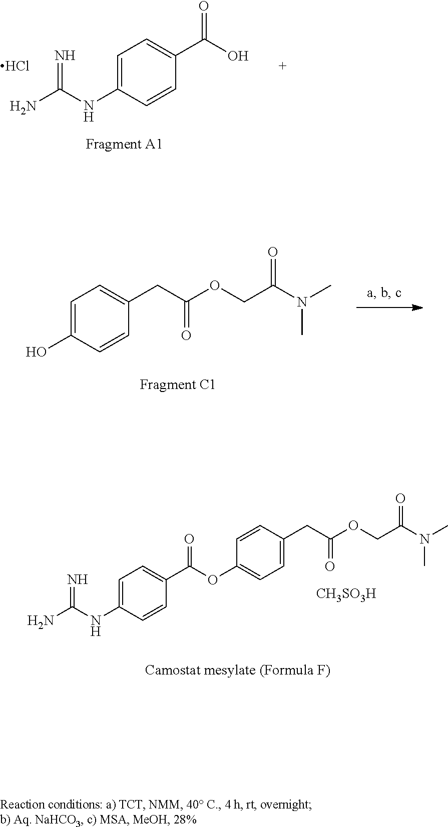

2,4,6-Trichloro-1,3,5-triazine (TCT) (0.250 g, 1.39 mmol) and 4-methylmorpholine (20 ml) were added in a round bottom flask and stirred at room temperature for 5 minutes. To the reaction mixture 4-guanidinobenzoic acid A1 (0.500 g, 2.79 mmol) was added and stirred at 40° C. for 4 hours. Then 2-(dimethylamino)-2-oxoethyl 2-(4-hydroxyphenyl) acetate C1 (0.662 g, 2.79 mmol) was added and the reaction mixture was stirred overnight at room temperature. Upon completion of the reaction i.e, formation of product D2 (checked by TLC), solvent was evaporated and purified by column chromatography. The product D2 was then dissolved in methanol and treated with methanesulfonic acid, upon adding diethyl ether Camostat mesylate (F) was precipitated. TLC (DCM/MeOH, 7:3) R_{f}=0.6; (0.75 g, yield 28%); white solid; m.p.: 153-158° C. ¹H NMR (400 MHz, DMSO-d⁶) δ 0 8.28-8.26 9 (d, J=8 Hz, 2H), 7.48-7.43 (m, 4H), 7.21-7.19 (d, J=8 Hz, 2H), 4.87 (s, 2H), 3.85 (s, 2H), 3.02 (s, 3H), 2.97 (s, 3H), 2.72 (s, 3H). ¹³C {¹H} NMR (101 MHz, CD₃OD) 8171.4, 167.6, 164.2, 156.2, 149.9, 140.4, 131.9, 131.4, 130.4, 127.2, 123.4, 121.3, 61.3, 39.2, 38.0, 34.7, 34.4, MS (ESI+) m/z calcd for C₂₀H₂₃N₄O₅ 399.16 (M+H); found 399.42.

### Example 9

### Synthesis of 4-(2-(2-(dimethylamino)-2-oxoethoxy)-2-oxoethyl)phenyl 4-guanidinobenzoate methanesulfonate (Camostat mesylate)

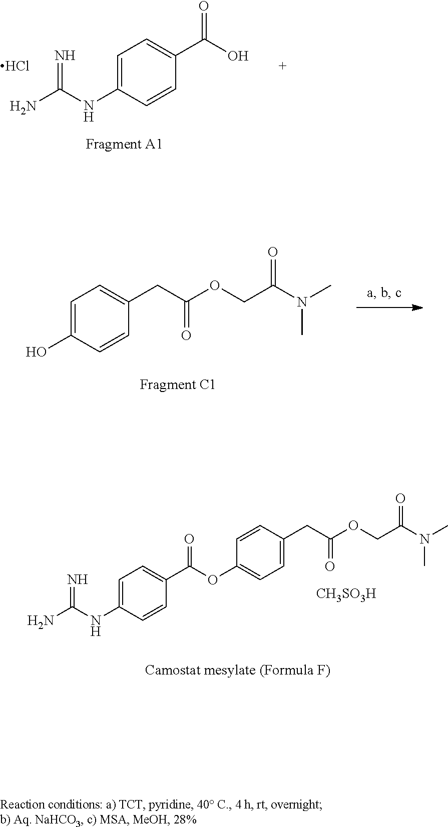

2,4,6-Trichloro-1,3,5-triazine (TCT) (0.250 g, 1.39 mmol) and 4-methylmorpholine (20 ml) were added in a round bottom flask and stirred at room temperature for 5 minutes. To the reaction mixture 4-guanidinobenzoic acid A1 (0.500 g, 2.79 mmol) was added and stirred at 40° C. for 4 hours. Then 2-(dimethylamino)-2-oxoethyl 2-(4-hydroxyphenyl) acetate C1 (0.662 g, 2.79 mmol) was added and the reaction mixture was stirred overnight at room temperature. Upon completion of the reaction i.e, formation of product D2 (checked by TLC), solvent was evaporated and purified by column chromatography. The product D2 was then dissolved in methanol and treated with methanesulfonic acid, upon adding diethyl ether Camostat mesylate (F) was precipitated (0.20 g, yield 23%).

### Example 10

### Synthesis of 4-(2-(2-(dimethylamino)-2-oxoethoxy)-2-oxoethyl)phenyl 4-guanidinobenzoate methanesulfonate (Camostat mesylate)

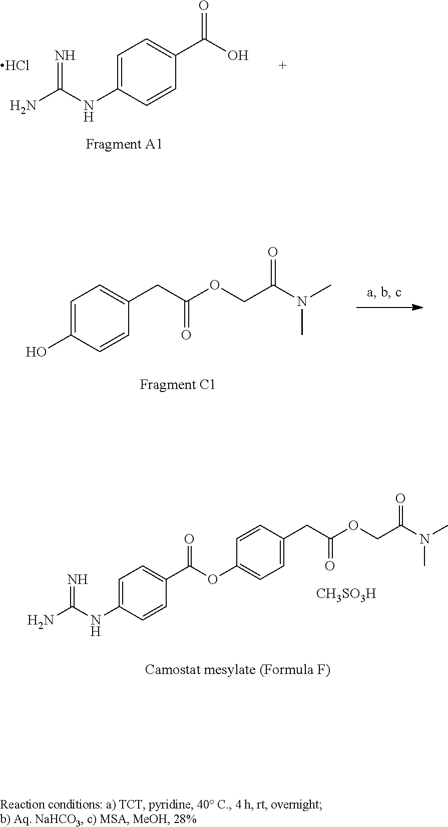

2,4,6-Trichloro-1,3,5-triazine (TCT) (0.250 g, 1.39 mmol) and 4-methylmorpholine (20 ml) were added in a round bottom flask and stirred at room temperature for 5 minutes. To the reaction mixture 4-guanidinobenzoic acid A1 (0.500 g, 2.79 mmol) was added and stirred at 40° C. for 4 hours. Then 2-(dimethylamino)-2-oxoethyl 2-(4-hydroxyphenyl) acetate C1 (0.662 g, 2.79 mmol) was added and the reaction mixture was stirred overnight at room temperature. Upon completion of the reaction i.e, formation of product D2 (checked by TLC), solvent was evaporated and purified by column chromatography. The product D2 was then dissolved in methanol and treated with methanesulfonic acid, upon adding diethyl ether Camostat mesylate (F) was precipitated (0.17 g, yield 20%).

### Example 11

### Synthesis of 4-(2-(2-(dimethylamino)-2-oxoethoxy)-2-oxoethyl)phenyl 4-guanidinobenzoate methanesulfonate (Camostat mesylate)

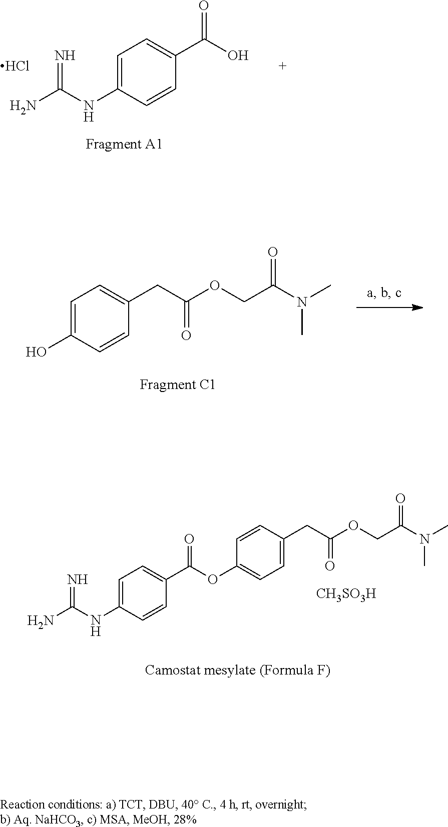

2,4,6-Trichloro-1,3,5-triazine (TCT) (0.250 g, 1.39 mmol) and 4-methylmorpholine (20 ml) were added in a round bottom flask and stirred at room temperature for 5 minutes. To the reaction mixture 4-guanidinobenzoic acid A1 (0.500 g, 2.79 mmol) was added and stirred at 40° C. for 4 hours. Then 2-(dimethylamino)-2-oxoethyl 2-(4-hydroxyphenyl) acetate C1 (0.662 g, 2.79 mmol) was added and the reaction mixture was stirred overnight at room temperature. Upon completion of the reaction i.e, formation of product D2 (checked by TLC), solvent was evaporated and purified by column chromatography. The product D2 was then dissolved in methanol and treated with methanesulfonic acid, upon adding diethyl ether Camostat mesylate (F) was precipitated (0.15 g, yield 17%).

### Example 12

### Synthesis of p-guanidinobenzoic Acid (A)

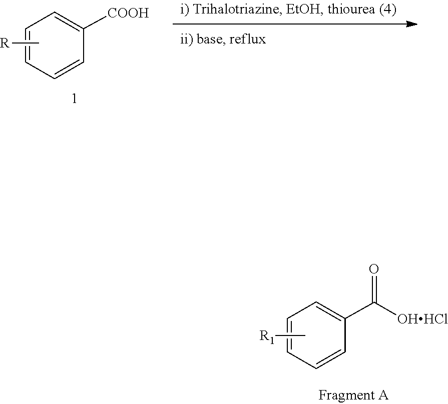

Thiourea (4) (12 g, 1 equiv.) and TCT (1 equiv.) in ethanol, were taken in a round bottom flask, and stirred for 2 hours, both reactants were consumed. Solvent was removed using rotary evaporator to obtain white solid. 1.5 g of the solid obtained was taken in a 10 mL round bottomed flask along with 0.699 g of p-aminobenzoic acid (1) and stirred for 15 min. In the reaction mixture, 3.56 mL N-methylmorpholine was added. After 1 hour both the reactants were consumed (TLC analysis) with the formation of 4-guanidinobenzoic acid A along with some other spots. N-methylmorpholine was removed under vacuum and product was isolated (0.15 g, yield 15%) using column chromatography.

### Advantages of Present Invention

1. The use of trichlorotriazine (TCT) as coupling reagent being inexpensive and stable provide advantages over the ones used in the prior art.
2. The present invention also provides ease of operation during work up in contrary to prior art and avoid repeated washing and treatment with solvents.
3. The use of trichlorotriazine (TCT) also provides the cost effective route for the synthesis of Nafamostat, Camostat and their derivatives.
4. Consumption of TCT as a coupling reagent make the preparation of Nafamostat or Camostat simple, high yielding, low impurities and suitable for industrial production.

1-7. (canceled)

8. A process for preparing a compound according to formula (F): 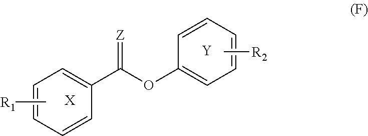
where:
Z is O, S, or NH;
rings X and Y are selected from group consisting of naphthalene, phenanthrene, quinoline, isoquinoline;
R¹ is selected from guanidinyl, amidinyl, 2-(dimethylamino)-2-oxoethyl acetyl, halogen, alkyl, amide, cyano, nitro, amino, methoxy, O-benzyl esters, N-benzyl esters, hydroxyl, aryl, or heteroaryl;
R² is selected from guanidinyl, amidinyl, 2-(dimethylamino)-2-oxoethyl acetyl, halogen, alkyl, amide, cyano, nitro, amino, methoxy, O-benzyl esters, N-benzyl esters, hydroxyl, aryl, heteroaryl, or substituted benzenes;
the process comprising:
(i) reacting 2,4,6-trihalo-1,3,5-triazine and a base at 25° C. to 40° C. for 5 minutes to obtain an activated complex;
(ii) reacting the activated complex obtained in (i) with fragment (A): 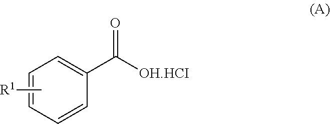
at 40° C. to 60° C. for 4 hours to obtain an intermediate compound;
(iii) reacting the intermediate compound obtained in (ii) with fragment (B): 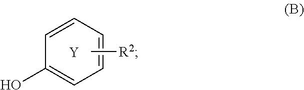
in a molar ratio from 0 to 1 and with fragment (C): 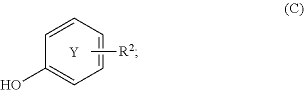
in a molar ratio from 0 to 1 to obtain a compound (D): 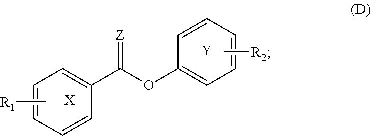
(iv) treating the compound D obtained in (iii) with aqueous sodium bicarbonate to produce a bicarbonate salt of the compound D;
(v) optionally purifying the bicarbonate salt of compound D obtained in (iv);
(vi) treating the compound D obtained in (iv) or (v) with methanesulfonic acid in the presence of methanol to provide the compound of formula (F).

9. The process according to claim 8, wherein the compound D is selected from Nafamostat mesylate or Camostat mesylate.

10. The process, as claimed in claim 8, further comprising preparing fragment A by reacting p-aminobenzoic acid with thiourea in presence of base in ethanol.

11. The process according to claim 8, wherein the base is selected from the group consisting of pyridine, N-methylmorpholine, trimethylamine, and diazabicycloundecene.

12. The process according to claim 8, wherein the base is N-methylmorpholine.

13. The process according to claim 8, wherein the trihalotriazine is selected from the group consisting of trichlorotriazine, tribromotriazine, and trifluorotriazine.

14. The process according to claim 8, wherein the base is selected from sodium carbonate, potassium carbonate, cesium carbonate, or ammonium carbonate.

15. The process according to claim 8, wherein the base is potassium carbonate.

16. The process according to claim 8, wherein the compound D is Nafamostat mesylate and a yield of the Nafamostat mesylate is from 54% to 70%.

17. The process according to claim 8, wherein the compound D is Camostat mesylate and a yield of the Camostat mesylate is from 17% to 28%.
